(19) United States
(12) Patent Application Publication
(10) Pub. No.: US 2025/0025315 A1
(43) Pub. Date: Jan. 23, 2025

(54) EXPANDABLE IMPLANT ASSEMBLY

(71) Applicant: LIFE SPINE, INC., Huntley, IL (US)

(72) Inventor: Daniel Predick, Wheat Ridge, CO (US)

(21) Appl. No.: 18/908,080

(22) Filed: Oct. 7, 2024

Related U.S. Application Data
(63) Continuation of application No. 18/108,402, filed on Feb. 10, 2023, now Pat. No. 12,138,178.
Application No. 18/108,402 is a continuation of application No. 16/850,795, filed on Apr. 16, 2020, now Pat. No. 11,602,439.

(51) Int. Cl. A61F 2/44; A61F 2/30

(52) U.S. Cl. CPC A61F 2/4425; A61F 2002/30578; A61F 2002/443; A61F 2250/0006; A61F 2250/0009

(57) ABSTRACT

An expandable implant. The expandable implant includes a base member having a top surface and a bottom surface opposite the top surface, and an adjustable member adjustably coupled to the base member and movable between a first, collapsed position, and a second, expanded position. The adjustable member has a top surface and a bottom surface opposite the top surface. The top surface of the adjustable member and the bottom surface of the base member form a first angle while the adjustable member is in the first, collapsed position, and the top surface of the adjustable member and the bottom surface of the base member form a second angle while the adjustable member is in the second, expanded position. The first angle is different from the second angle.

### CROSS REFERENCE TO RELATED APPLICATIONS

This application is a continuation of U.S. patent application Ser. No. 18/108,402, filed Feb. 10, 2023, which is a continuation of U.S. patent application Ser. No. 16/850,795, filed Apr. 16, 2020, the disclosures of which are incorporated herein by reference.

### BACKGROUND

The present disclosure relates to expandable implants and devices, including spinal interbody and intravertebral body devices, and vertebral interbody and intravertebral devices that are expandable after spinal placement thereof.

Many people contend with spine issues as a result of age, disease, and trauma, as well as congenital and acquired complications and conditions. While some of these issues can be alleviated without surgery, other issues necessitate surgery. Spinal fusion may be recommended for conditions such as spondylolistheses, degenerative disc disease, or recurrent disc herniation, and is designed to create solid bone between adjacent vertebrae, thereby eliminating any movement between the bones. A spinal fusion uses an implant or device known as an interbody cage or spacer along with bone graft and/or bone graft substitute that is inserted into the disc space between adjacent vertebrae from one side of the spine. Typically, additional surgical hardware (implants) such as pedicle screws and rods or plates are attached to the back of the vertebrae. As the bone graft heals, it fuses the adjacent vertebrae to form one long vertebra.

Fusion cages, as well as other types of implants, bodies and/or devices, are frequently utilized in spinal surgery inside a vertebra (intravertebral) and/or between vertebrae of a patient (interbody), or adjacent other bone bodies. With interbody devices, one or more such spinal bodies are placed between vertebrae to provide support and promote fusion between adjacent vertebrae where such is necessary due to disease, injury, general deterioration or congenital problem. With intravertebral devices, one or more spinal bodies are placed within a vertebra.

Spinal devices, such as fusion cages and/or the like, are inserted into a spinal space either anteriorly, posteriorly, laterally or posterolaterally.

### SUMMARY

In some embodiments, an expandable implant is disclosed. The expandable implant includes a base member having a top surface and a bottom surface opposite the top surface, and an adjustable member adjustably coupled to the base member and movable between a first, collapsed position, and a second, expanded position. The adjustable member has a top surface and a bottom surface opposite the top surface. The top surface of the adjustable member and the bottom surface of the base member form a first angle while the adjustable member is in the first, collapsed position, and the top surface of the adjustable member and the bottom surface of the base member form a second angle while the adjustable member is in the second, expanded position. The first angle is different from the second angle.

In further embodiments, an expandable implant is disclosed. The expandable implant includes a base member having a guide groove and an adjustable member adjustably coupled to the base member and movable between a first, collapsed position, and a second, expanded position, wherein the adjustable member has a guide rail. The guide groove is configured to receive the guide rail, such that the guide rail translates within the guide groove as the adjustable member is moved from the first, collapsed position to the second, expanded position. The guide rail has a curvature such that the adjustable member moves in a non-linear manner from the first, collapsed position to the second, expanded position.

In further embodiments, an expandable implant is disclosed. The expandable implant includes a plurality of anchoring members, a base member configured to receive an anchoring member, an adjustable member adjustably coupled to the base member and movable between a first, collapsed position, and a second, expanded position, wherein the adjustable member is configured to receive an anchoring member, and a control assembly including a control shaft.

Rotation of the control shaft causes relative movement of the adjustable member relative to the base member.

### BRIEF DESCRIPTION OF THE FIGURES

The features of the subject matter disclosed herein will be better understood by reference to the accompanying drawings which illustrate the subject matter disclosed herein, wherein:

FIG. 1 is a perspective view of an implant in a collapsed position according to an example embodiment.

FIG. 2 is another perspective view of the implant of FIG. 1 in a collapsed position according to an example embodiment.

FIG. 3 is a top view of the implant of FIG. 1 in a collapsed position according to an example embodiment.

FIG. 4 is a front view of the implant of FIG. 1 in a collapsed position according to an example embodiment.

FIG. 5 is a perspective view of the implant of FIG. 1 in an expanded position according to an example embodiment.

FIG. 6 is another perspective view of the implant of FIG. 1 in an expanded position according to an example embodiment.

FIG. 7 is another perspective view of the implant of FIG. 1 in an expanded position according to an example embodiment.

FIG. 8 is a rear view of the implant of FIG. 1 in an expanded position according to an example embodiment.

FIG. 9 is an exploded view of the implant of FIG. 1 according to an example embodiment.

FIG. 10 is a perspective view of an adjustable member of the implant of FIG. 1 according to an example embodiment.

FIG. 11 is another perspective view of the adjustable member of FIG. 10 according to an example embodiment.

FIG. 12 is a perspective view of the underside of the adjustable member of FIG. 10 according to an example embodiment.

FIG. 13 is a bottom view of the adjustable member of FIG. 10 according to an example embodiment.

FIG. 14 is a perspective view of a control shaft and two control members of the implant of FIG. 1 according to an example embodiment.

FIG. 15 is a perspective view of the control shaft of FIG. 14 according to an example embodiment.

FIG. 16 is a perspective view of a control member of the implant of FIG. 1 according to an example embodiment.

FIG. 17 is a perspective view of a control member of the implant of FIG. 1 according to an example embodiment.

FIG. 18 is a perspective view of a control shaft and control members within a base member of the implant of FIG. 1 according to an example embodiment.

FIG. 19 is a perspective view of a base member of the implant of FIG. 1 according to an example embodiment.

FIG. 20 is a rear perspective view of the base member of FIG. 19 according to an example embodiment.

FIG. 21 is a perspective view of an implant according to another example embodiment.

FIG. 22 is a perspective view of the implant of FIG. 21 according to an example embodiment.

FIG. 23 is a side view of the implant of FIG. 21 according to an example embodiment.

FIG. 24 is a front view of the implant of FIG. 21 according to an example embodiment.

FIG. 25 is a front view of a control shaft and cam screw according to an example embodiment.

FIG. 26 is a front view of a control shaft and cam screw of the implant of FIG. 21 according to an example embodiment.

FIGS. 27A-B are schematic representations of a control mechanism according to an example embodiment.

Corresponding reference characters indicate corresponding parts throughout the several views. Although the drawings represent embodiments of the disclosure, the drawings are not necessarily to scale and certain features may be exaggerated in order to better illustrate and explain the principles of the present disclosure. The exemplifications set out herein illustrate several embodiments, but the exemplifications are not to be construed as limiting the scope of the disclosure in any manner.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The present disclosure relates to expandable and/or dynamic implants. In an example embodiment, the implant may be an interbody (between adjacent vertebrae), intravertebral-body (inside the vertebrae) and/or spinal stabilization devices that may or may not be used as interbody fusion cages or devices, interbody/intravertebral bodies/body stabilization devices and/or the like (e.g., spinal device(s)) for providing support, stabilization and/or promoting bone growth between or inside vertebrae or other portions of bone that have been destabilized or otherwise due to injury, illness and/or the like. Particularly, the present disclosure provides various versions of dynamic (expandable and/or expandable and retractable) interbody/intravertebral body devices that are usable in a spinal column or other areas of a human.

Various embodiments disclosed herein are directed to expandable implants that are implantable between adjacent bodies of bone. For example, the implant may be implanted or inserted into a human spine adjacent upper and lower vertebrae of the spine. According to various exemplary embodiments, the components of the implants disclosed herein may be made of any suitable material(s), including a variety of metals, plastics, composites, or other suitable bio-compatible materials. In some embodiments, one or more components of the implants disclosed herein may be made of the same material, while in other embodiments, different materials may be used for different components of the various implants.

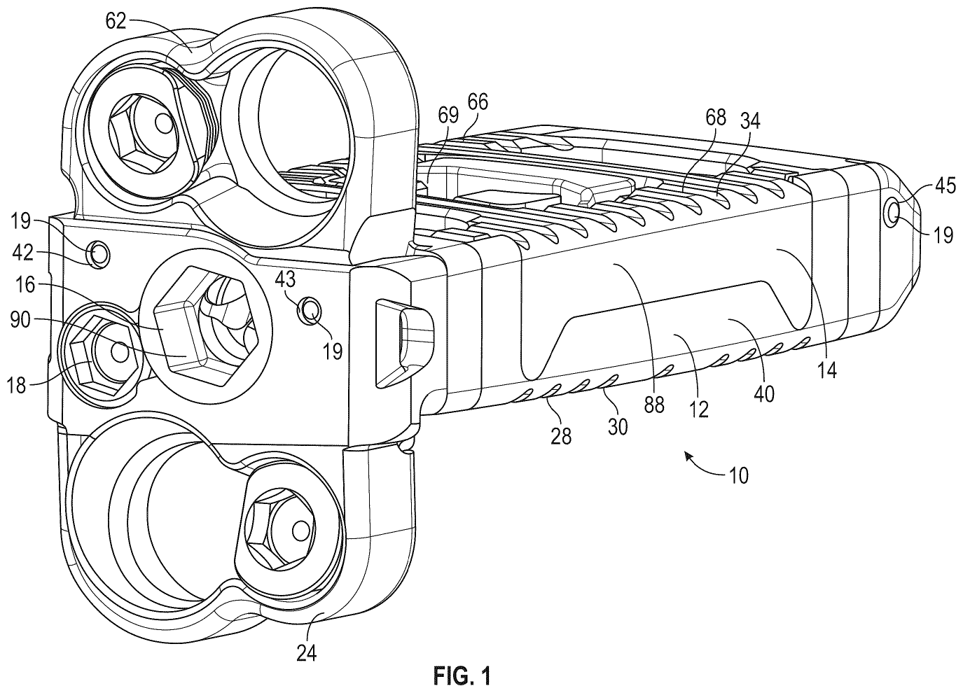

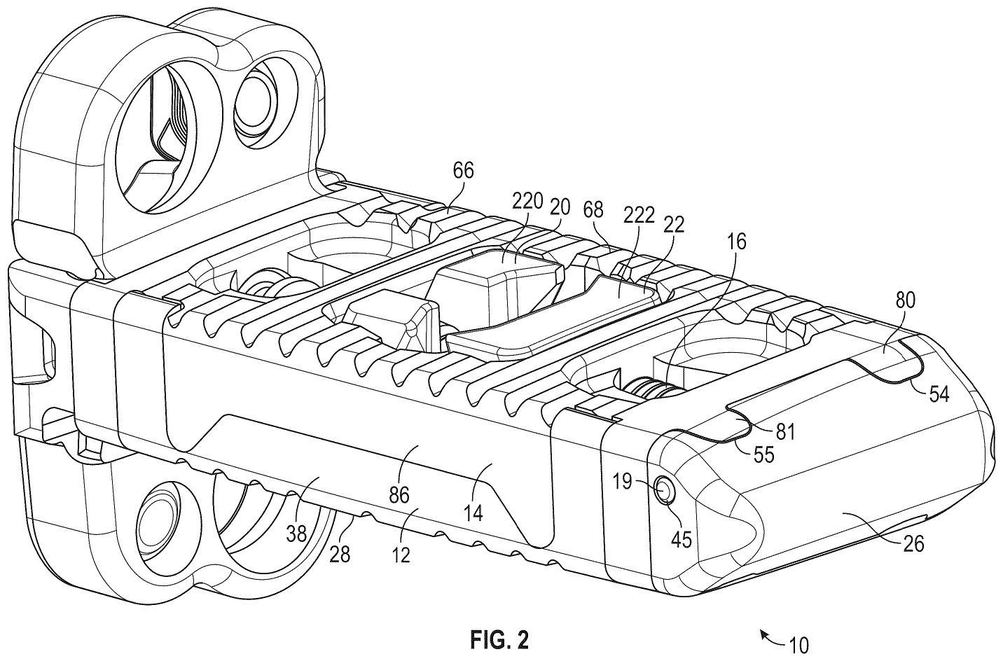

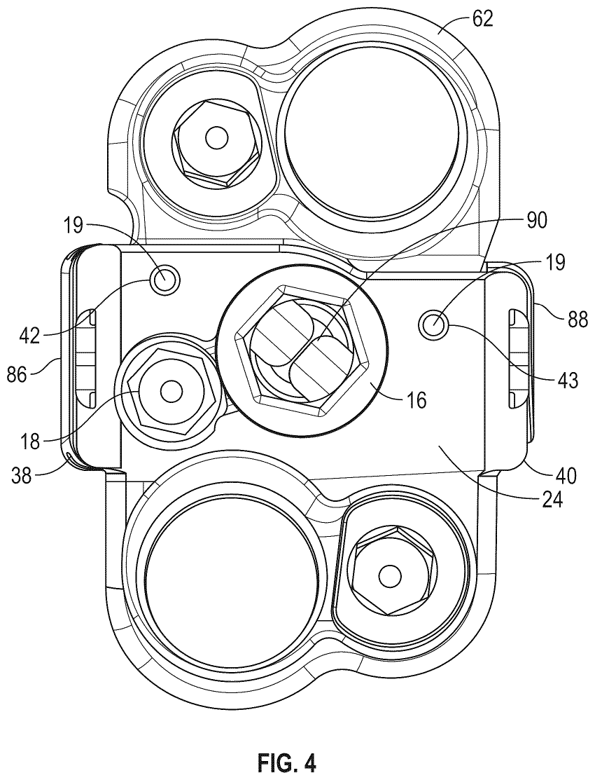

Referring now to FIGS. 1-4, an expandable implant 10 is shown according to an exemplary embodiment. The implant 10 is usable, for example, between and/or within vertebral bodies of the spine. It should be understood that the implant 10 may in some embodiments be usable in other portions of the body in addition to the spine, and all such applications are to be understood to be within the scope of the present disclosure.

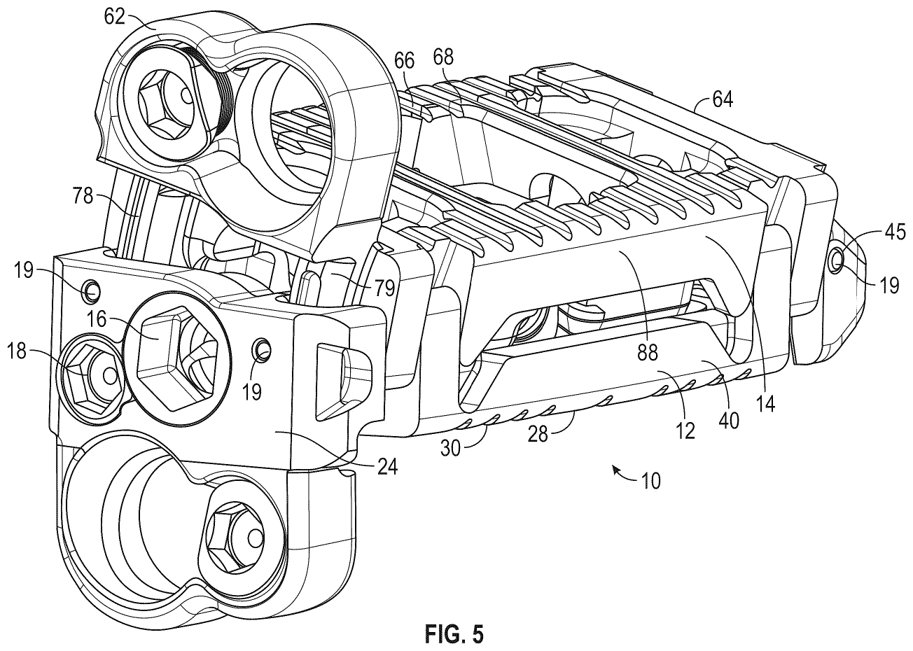

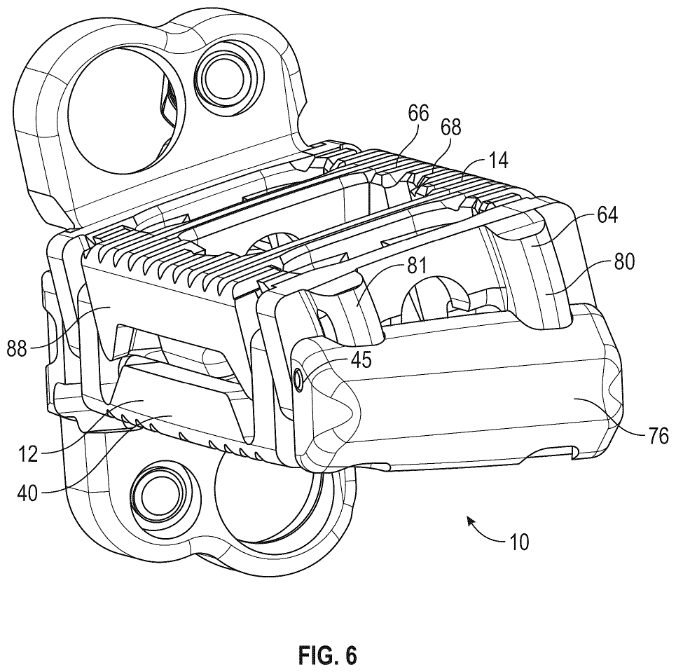

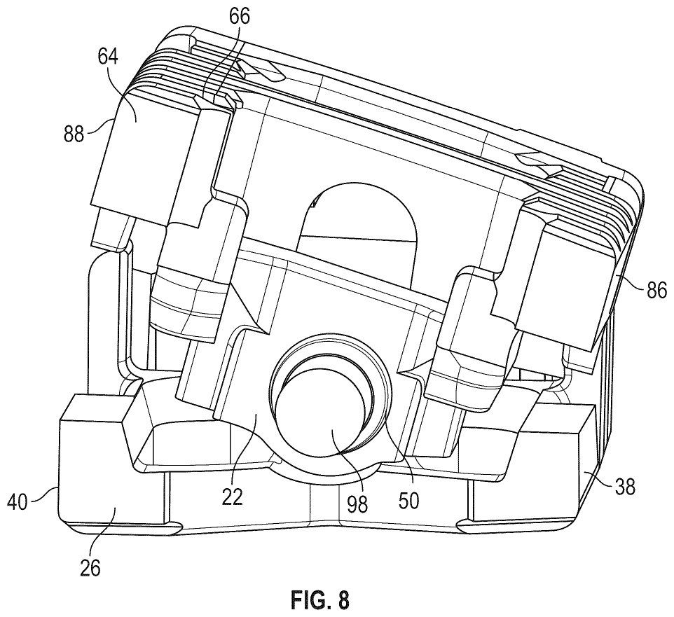

According to some embodiments, the implant 10 includes a base member 12 and an adjustable member 14 adjustably coupled to the base member 12. The implant 10 may further include a control shaft 16 received by the base member 12 retained by a cam screw 18 passing through a portion of the base member 12. A first control member 20 and a second control member 22 are received on the control shaft 16 and are movable along the control shaft 16 to adjust a position of the adjustable member 14 between a collapsed position, as shown in FIGS. 1-4, and an expanded position, as shown in FIGS. 5-8.

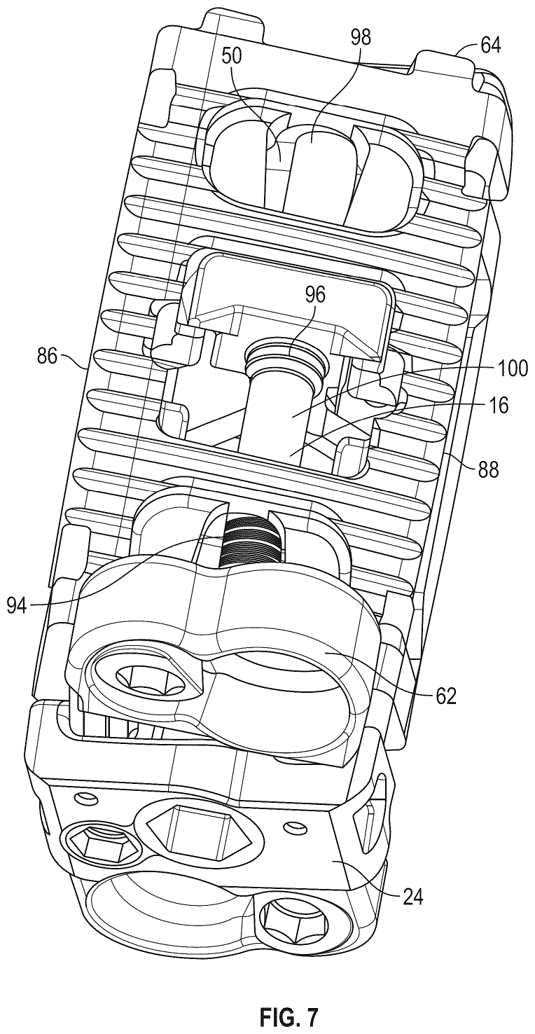

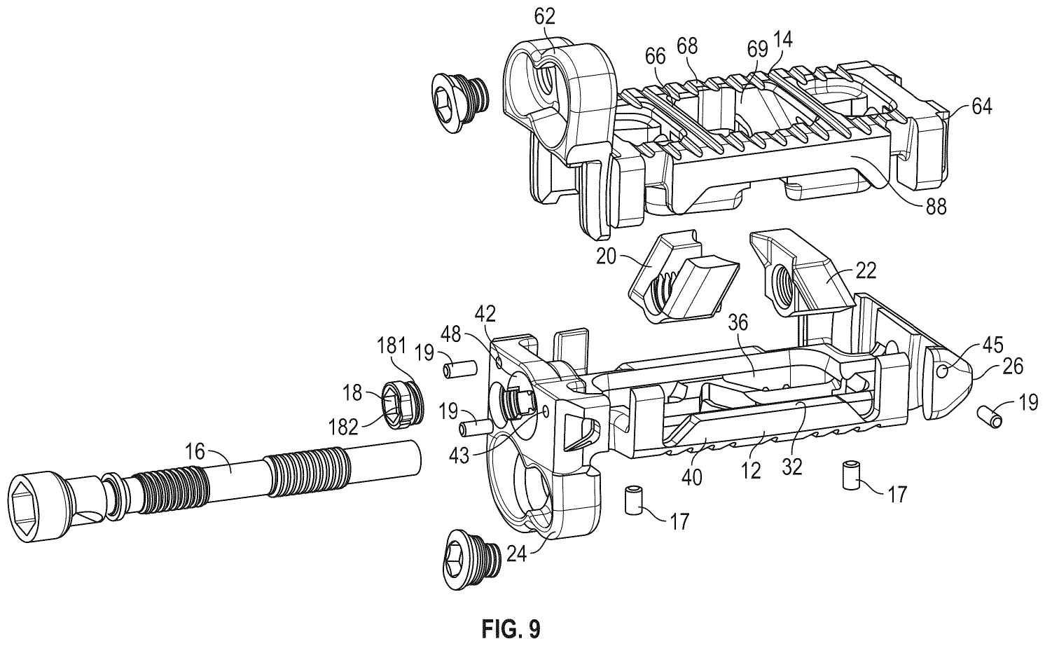

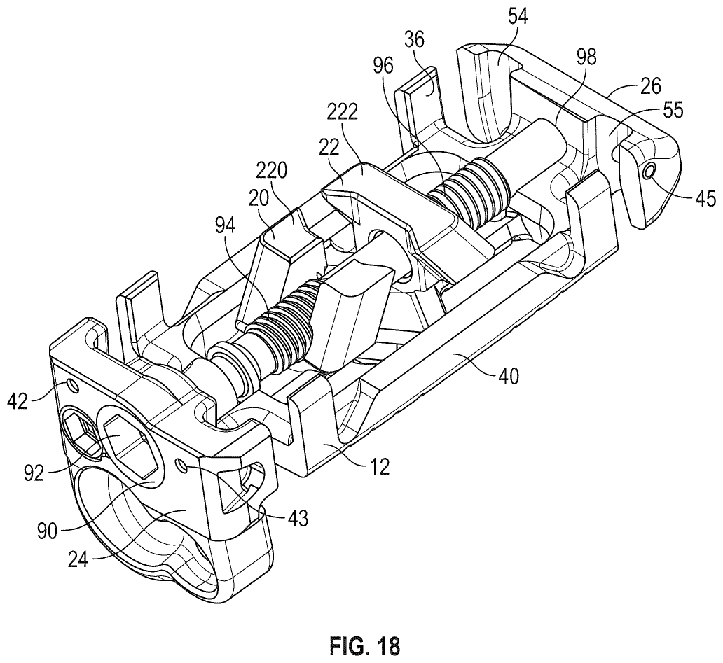

As shown in FIG. 18, the base member 12 includes a front or first end 24, a rear or second end 26, and a central cavity 36 disposed between the first end 24 and the second end 26. The base member 12 further includes a bottom surface 28 having ridges or projections 30 formed by corresponding grooves, a top surface 32 opposite the bottom surface 28, a first side 38, and a second side 40. The projections 30 are configured to engage adjacent portions of bone. In some embodiments, the front end 24 includes a first pin aperture 42 configured to receive a retention pin 19 and a second pin aperture 43 configured to receive a retention pin 19. In some embodiments, the first side 38 includes a third pin aperture, and the second side 40 includes a fourth pin aperture 45 configured to receive a retention pin 19. In some embodiments, such as the embodiment shown in FIG. 1, the first side 38 does not include a third pin aperture. The first pin aperture 42, second pin aperture 43, third pin aperture, and fourth pin aperture 45 may be configured to individually receive a retention pin 19 (e.g. in a press fit or other manner of retention). The first end 24 of the base member 12 includes a control bore 48 (see FIG. 9) configured to receive a first portion of the control shaft 16. The second end 26 includes a tip bore 50 (see FIG. 7) configured to receive a head 90 of the control shaft 16.

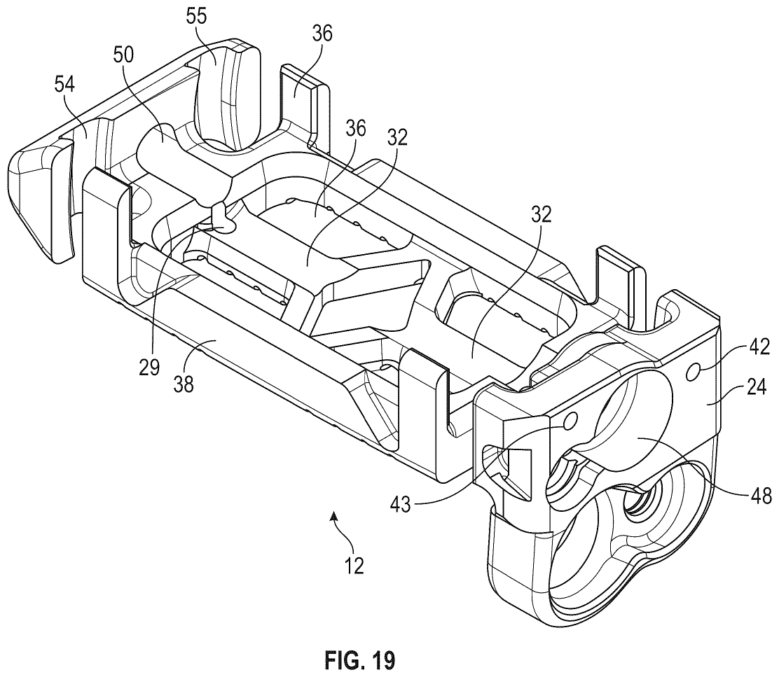

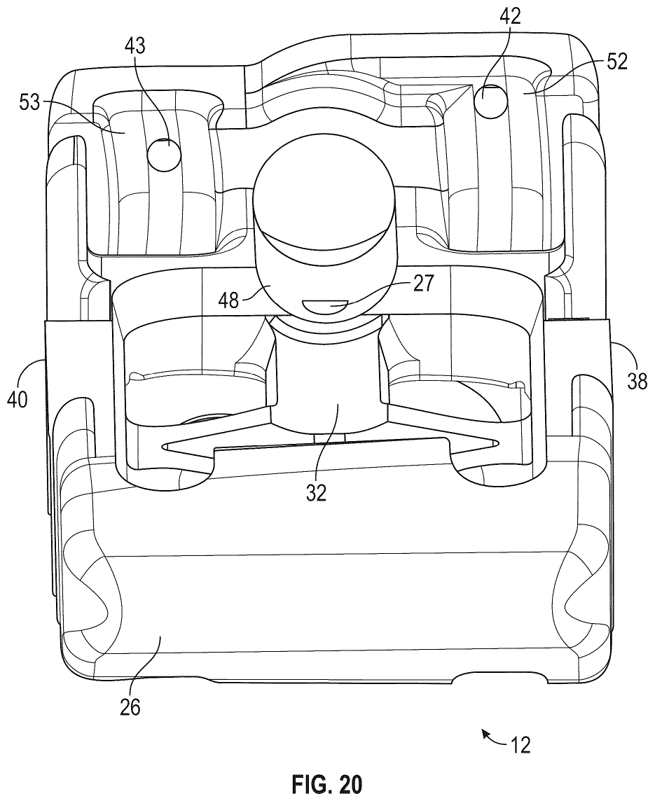

In further embodiments, the base member 12 includes a first support pin aperture 27, shown in FIG. 20, and a second support pin aperture 29, shown in FIG. 19. The first support pin aperture 27 and second support pin aperture 29 may be individually configured to receive a support pin 17 (e.g. in a press fit or other manner of retention). The support pin 17 may extend into the central cavity 36 and may support the control shaft 16 such that the control shaft 16 will not bottom out against the top surface 32 of the base member 12.

In further embodiments, the base member 12 may include a first front guide groove 52, a second front guide groove 53, a first rear guide groove 54, and a second rear guide groove 55. The first front guide groove 52, second front guide groove 53, first rear guide groove 54, and second rear guide groove 55 may be utilized to customize the expansion profile of the implant 10, as will be discussed in further detail.

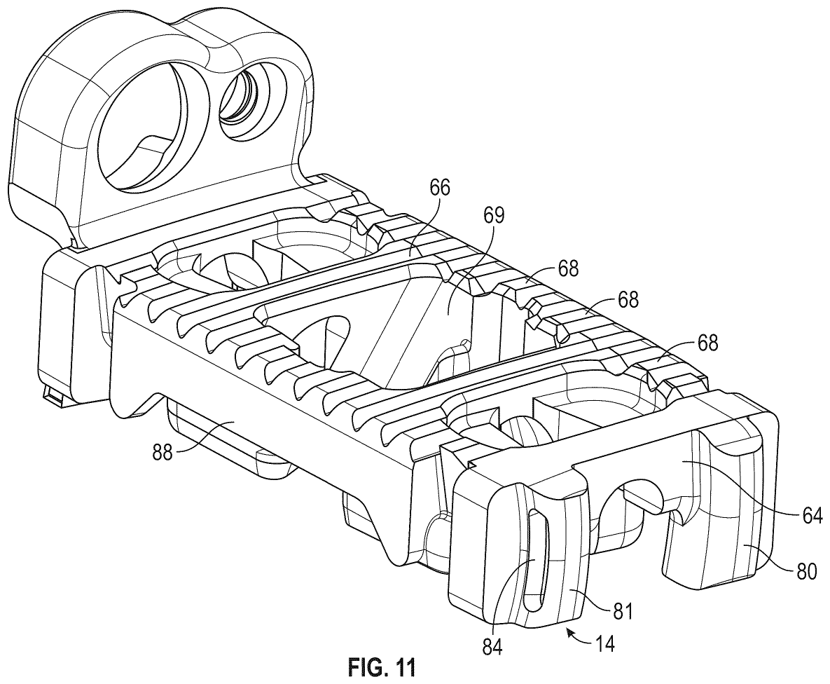

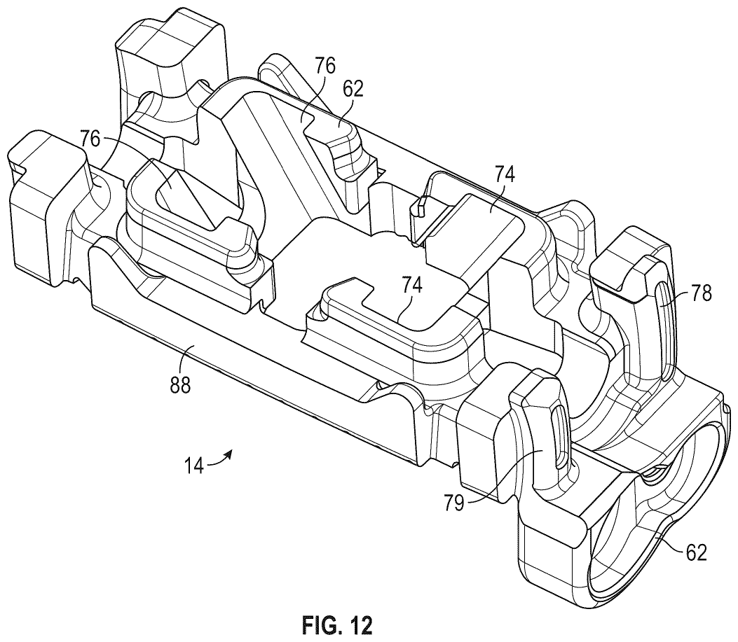

In some embodiments, as shown in FIGS. 11 and 12, the adjustable member 14 includes a front or first end 62, a rear or second end 64, and a central recess or cavity 69 positioned between the first end 62 and the second end 64. The adjustable member 14 further includes a top surface 66 having ridges or projections 68 formed by corresponding grooves and a bottom surface 67. The adjustable member 14 also includes a first side portion 86, and a second side portion 88. In some embodiments, the first and second side portions 86, 88 have a shape generally corresponding to the shape of the first side 38 and the second side 40 of the base member 12. In other embodiments, the first and second side portions 86, 88 have shapes differing from the shapes of the first side 38 and second side 40 of the base member 12.

As shown in FIG. 5, in further embodiments, the adjustable member 14 may include a first front guide rail 78, a second front guide rail 79, a first rear guide rail 80, and a second rear guide rail 81. In some embodiments, the first front guide rail 78 is configured to be received by the first front guide groove 52, the second front guide rail 79 is configured to be received by the second front guide groove 53, the first rear guide rail 80 is configured to be received by the first rear guide groove 54, and the second rear guide rail 81 is configured to be received by the second rear guide groove 55. According to an example embodiment, when the implant 10 expands (e.g. the adjustable member 14 moves in a direction away from the base member 12), the guide rails 78, 79, 80, 81 will individually translate within each respective guide groove 52, 53, 54, 55.

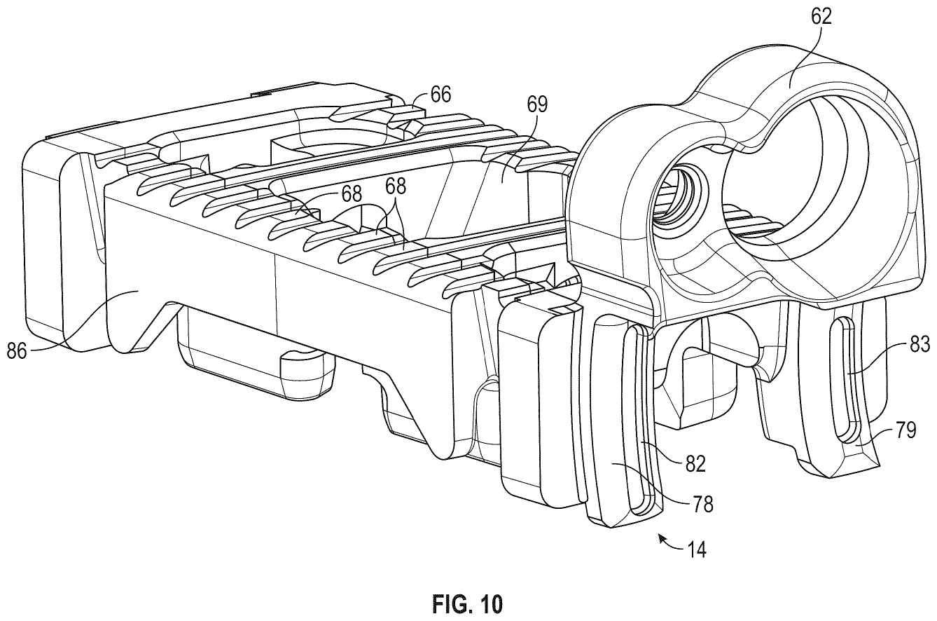

In further embodiments, such as the embodiment shown in FIG. 10, the first front guide rail 78 may include a first pin slot 82 configured to receive a retention pin 19. Further, the second front guide rail 79 may include a second pin slot 83 configured to receive a retention pin 19. Further, the first rear guide rail 80 may include a third pin slot configured to receive a retention pin 19. In some embodiments, such as the embodiment shown in FIG. 1, the first guide rail 80 does not include a third pin slot. Further, the second rear guide rail 81 may include a fourth pin slot 84 configured to receive a retention pin 19.

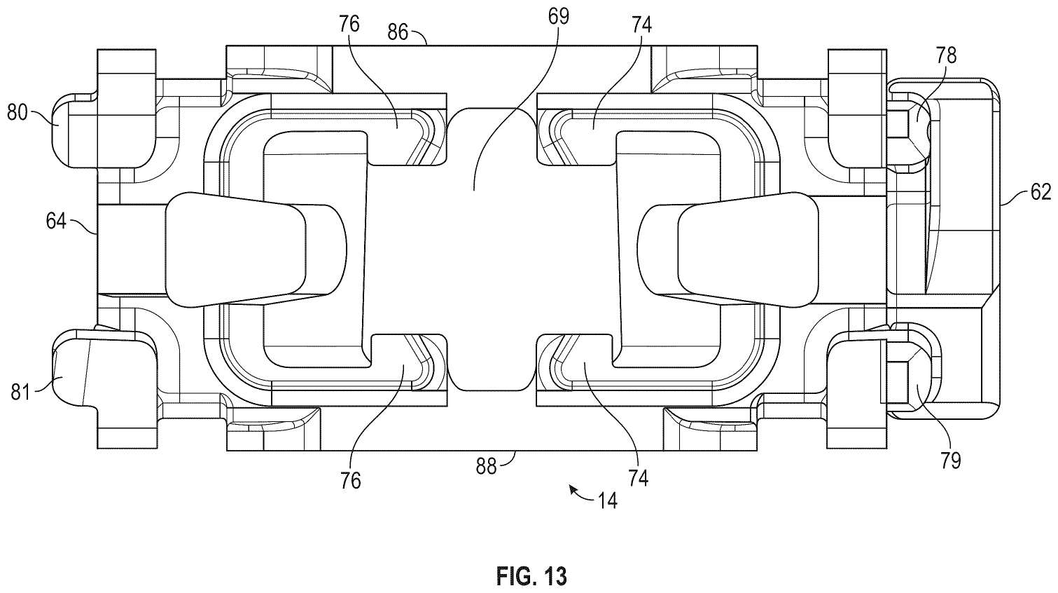

Referring to FIGS. 10-13, in some embodiments, the adjustable member 14 includes one or more control channels, such as a first control channel 74 and a second control channel 76. The first control channel 74 receives the first control member 20, and the second control channel 76 receives the second control member 22. In some embodiments, the control members 20, 22 are received in the control channels 74, 76 in a sliding manner such that the control members 20, 22 are able to translate within the control channels 74, 76. In further embodiments, each control channel has a shape such that the control channel surrounds the control member and at least partially corresponds in shape to the control member.

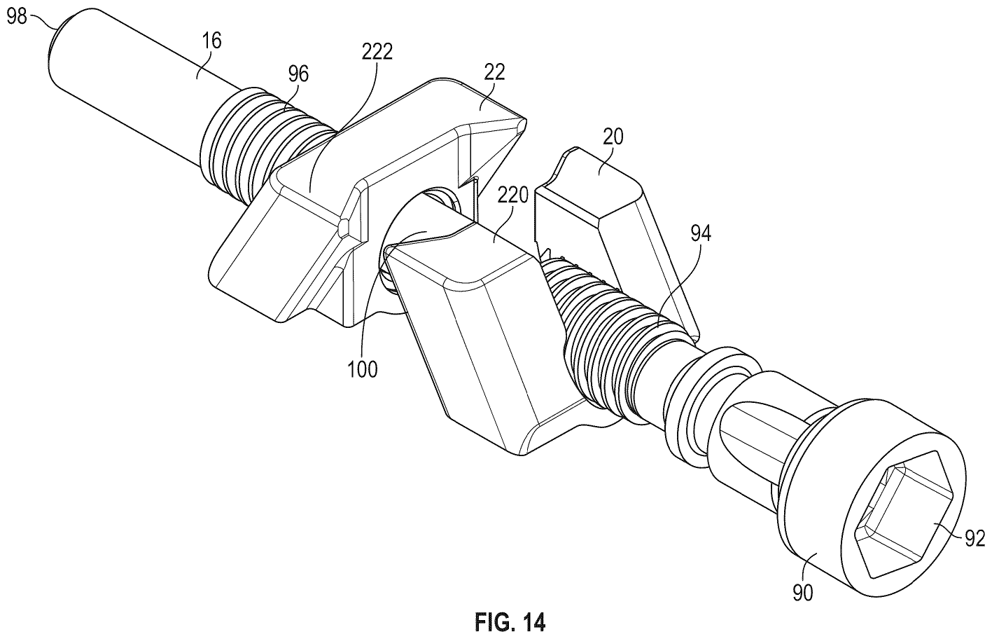

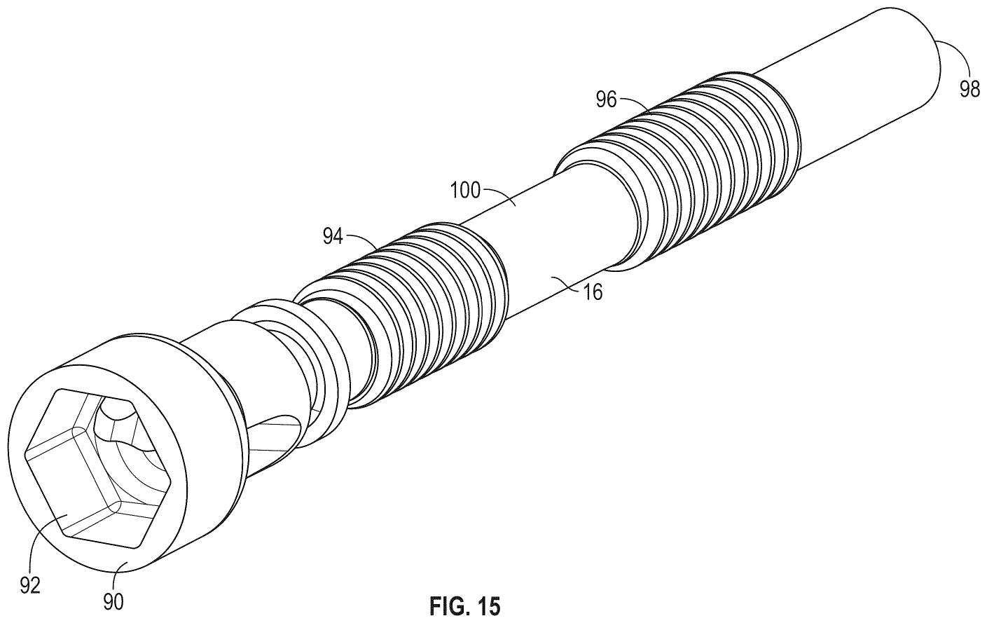

Referring to FIGS. 14 and 15, the control shaft 16 includes a head portion 90, a tool port 92 disposed within the head portion 90, and a tip 98 located at an end opposite the head portion 90. In some embodiments, the tip 98 is flat, while in other embodiments, the tip 98 may be pointed or beveled. In some embodiments, the control shaft 16 further includes a first control thread 94 and a second control thread 96. A non-threaded portion 100 may be located between the first control thread 94 and the second control thread 96.

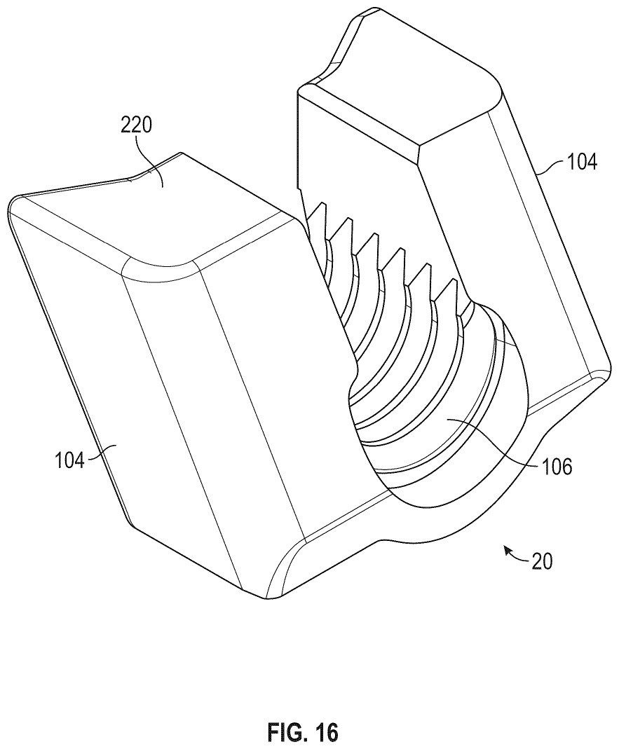

As shown in FIG. 16, the first control member 20 includes a top portion 220, one or more flat portions 104, and an internal thread 106. In some example embodiments, the first control member 20 is configured to be received by the first control channel 74, such that the flat portions 104 engage with the walls of the first control channel 74.

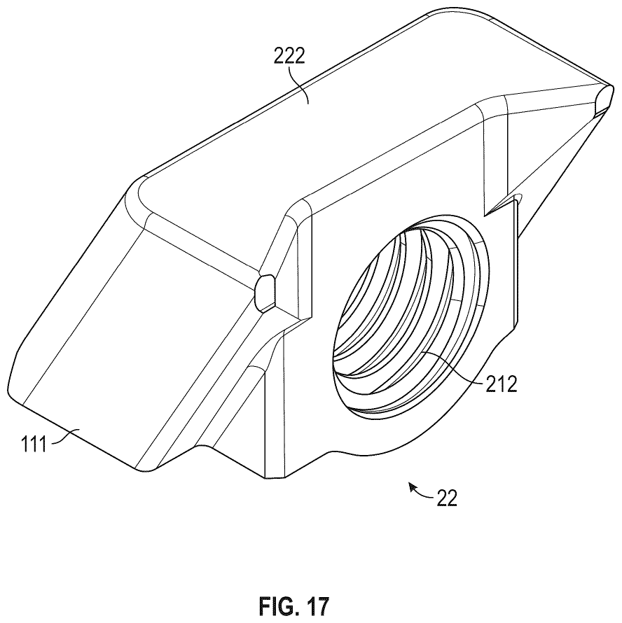

As shown in FIG. 17, the second control member 22 includes a top portion 220, one or more flat portions 111, and an internal thread 212. In some example embodiments, the second control member 22 is configured to be received by the second control channel 76, such that the flat portions 111 engage with the walls of the second control channel 76. In some example embodiments, the first control member 20 and the second control member 22 move or translate along the control shaft 16 and within or on the first control channel 74 and the second control channel 76, as will be described further herein.

Referring back to FIGS. 1-8, in some embodiments, the implant 10 is movable between at least a first, collapsed position, as shown in FIGS. 1-4, and a second, expanded position, shown in FIGS. 5-8. In the first position, the control shaft 16 is received by the control bore 48 and positioned within the central cavity 36 of the base member 12. In some embodiments, the first side 38 and the second side 40 of the base member 12 receive the first side 86 and the second side 88 of the adjustable member when the implant 10 is in the collapsed position, such that the projections and recesses have a relatively close fit to enable proper alignment between the adjustable member 14 and the base member 12, as shown in FIGS. 1-4. In other embodiments, the projections and recesses have a relatively loose fit to enable a desired angular offset between the adjustable member 14 and the base member 12.

In some embodiments, the control shaft 16 is received by the base member 12 such that the head 90 is positioned within the control bore 48 (see FIG. 9), the first control thread 94 and the second control thread 96 are positioned within the central cavity 36, and the head portion 90 is positioned within the tip bore 50 of the second end 26 of the base member 12. In some embodiments, the control shaft 16 is rotatable within the base member 12. The first control member 20 is received on the first control thread 94 of the control shaft 16, and the second control member 22 is received on the second control thread 96 of the control shaft 16.

In some embodiments, such as the embodiment shown in FIG. 14, the first control thread 94 and the second control thread 96 are threaded in opposite manners (e.g., left-handed and right-handed), such that upon rotation of the control shaft 16, the control members 20, 22 move in opposite directions along the control shaft 16. For example, the control shaft 16 may be configured such that rotation of the control shaft 16 in a first direction (e.g., clockwise) causes the first and second control members 20, 22 to move toward each other, and rotation of the control shaft 16 in a second direction (e.g., counter-clockwise) causes the first and second control member 20, 22 to move away from each other.

As the control members 20, 22 move along the control shaft 16, the control members 20, 22 further move within the control channels 74, 76, thereby causing relative movement of the adjustable member 14 and the base member 12. As the control members 20, 22 translate along the control shaft 16, the adjustable member 14 is moved upward or downward due to the angled shape of the first and second control channels 74, 76. Assuming a constant turn rate of the control shaft 16, the rate of movement of the control members 20, 22, and therefore the adjustable member 14, can be adjusted by modifying the slope of the control channels 74, 76 relative to the control shaft 16.

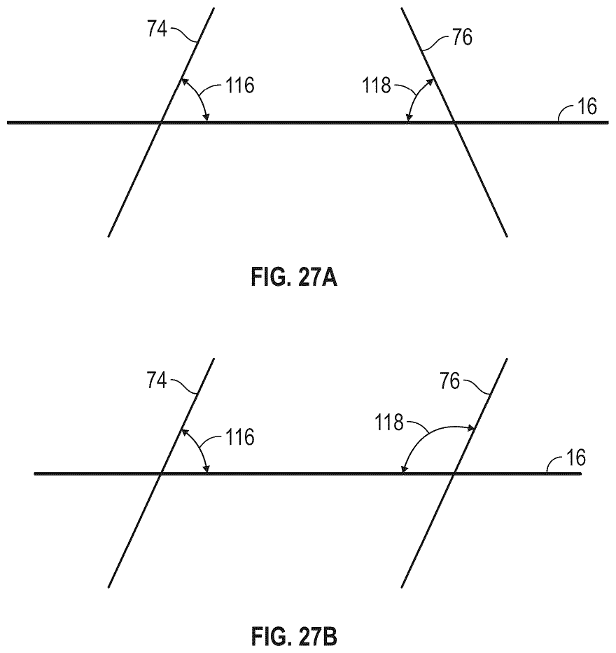

For example, referring to FIGS. 27A-C, schematic representations of the control shaft 16, the first control channel 74, and the second control channel 76 are shown according to various alternative embodiments. The first control channel 74 extends at a first angle 116 relative to the control shaft 16, and the second control channel 76 extends at a second angle 118 relative to the control shaft 16. The first and second angles 116, 118 define the rate at which the first control member 20 and the second control member 22 cause corresponding movement (e.g., expansion) of the first and second ends 62, 64 of the adjustable member 14 relative to the base member 12. As shown in FIG. 27A, in some embodiments, the first angle 116 and second angle 118 are approximately the same, and the control channels 74, 76 define linear paths, such that the rates of movement of the first and second ends 62, 64 of the adjustable member 14 are substantially the same and constant (assuming a constant rate of rotation of the control shaft 16). As shown in FIG. 27B, in some embodiments, rather than being angled toward each other in an upward direction, the first and second control channels 74, 76 may extend in a parallel manner or be configured to extend upward at angles in the same general direction.

Providing differing configurations for the first control channel 74 and the second control channel 76 enables customization of the characteristics of the implant 10 in the second, expanded position. For example, the control channels 74, 76 may be configured such that in a fully expanded position of the implant 10, one of the first end 62 and the second end 64 of the adjustable member 14 is expanded to a greater degree than the opposing end. Other configurations of the first and second control channels 74, 76 are possible according to various alternative embodiments.

In further embodiments, characteristics of the implant 10 in the second, expanded position may be further customized using at least one guide rail. For example, the implant 10 according to the example embodiment shown in FIGS. 5-8 has an angular expansion profile, wherein the top surface 66 of the adjustable member 14 and the bottom surface 28 of the base member 12 are not substantially parallel, but instead form an angle. The angle that the top surface 66 of the adjustable member 14 and the bottom surface 28 of the base member 12 form when the implant 10 is in the expanded position may be customized based on patient needs. For example, when the implant 10 is installed into a patient's spine, the angle that the top surface 66 of the adjustable member 14 and the bottom surface 28 of the base member 12 form when the implant 10 is in the expanded position may vary depending on the curvature of the patient's spine at the location the implant 10 is installed.

According to an example embodiment, an angular expansion profile of the implant 10 may be achieved using at least one guide rail and at least one control member. For example, in the example embodiment shown in FIGS. 5-8, the control shaft 16 is received by the base member 12 such that the head 90 is positioned within the control bore 48, the first control thread 94 and the second control thread 96 are positioned within the central cavity 36, and the end portion 98 is positioned within the tip bore 50 of the second end 26 of the base member 12 (see FIG. 7). In this embodiment, the control shaft 16 is rotatable within the base member 12. The first control member 20 is received on the first control thread 94 of the control shaft 16, and the second control member 22 is received on the second control thread 96 of the control shaft 16.

In this example embodiment, the first control thread 94 and the second control thread 96 are threaded in opposite manners (e.g., left-handed and right-handed), such that upon rotation of the control shaft 16, the control members 20, 22 move in opposite directions along the control shaft 16. In this example embodiment, the control shaft 16 is configured such that rotation of the control shaft 16 in a first direction (e.g., clockwise) causes the first and second control members 20, 22 to move toward each other, and rotation of the control shaft 16 in a second direction (e.g., counter-clockwise) causes the first and second control member 20, 22 to move away from each other.

As the control members 20, 22 move along the control shaft 16, the control members 20, 22 further move within the control channels 74, 76, thereby causing relative movement of the adjustable member 14 and the base member 12. As the control members 20, 22 translate along the control shaft 16, the adjustable member 14 is moved upward or downward due to the angled shape of the first and second control channels 74, 76. The rate of movement of the control members 20, 22 and the adjustable member 14 can therefore be adjusted by modifying the slope of the control channels 74, 76 relative to the control shaft 16.

When the control shaft 16 is turned in a second direction (e.g. counter-clockwise), the control members 20, 22 translate along the control shaft 16 away from each other. In doing so, the control members 20, 22 also translate within the control channels 74, 76 causing the adjustable member 14 to generally move away from the base member 12. Further, when the adjustable member 14 moves away from the base member 12, the guide rails 78, 79, 80, 81 individually translate within each respective guide groove 52, 53, 54, 55. In some embodiments, the guide rails 78, 79, 80, 81 and the guide grooves 52, 53, 54, 55 may run perpendicular to the top surface 66 of the adjustable member 14, thereby causing the adjustable member 14 to move upwards in a linear manner. In these example embodiments, the top surface 66 of the adjustable member 14 will be substantially parallel to the bottom surface 28 of the base member 12 when the implant 10 is in a second, expanded position. In other embodiments, such as the embodiments shown in FIGS. 5-8, the guide rails 78, 79, 80, 81 and the guide grooves 52, 53, 54, 55 may have a curvature. The curvature of the guide rails 78, 79, 80, 81 and the guide grooves 52, 53, 54, 55 will cause the adjustable member 14 to move upwards in a non-linear manner. Therefore, the implant 10 will have an angular expansion profile (e.g. the top surface 66 of the adjustable member 14 and the bottom surface 28 of the base member 12 are not substantially parallel, but instead form an angle).

In some embodiments, the first front guide rail 78 may have a larger radius (e.g. the radius of the circular arc which best approximates the curve of the guide rail at that point) than the second front guide rail 78 and the first rear guide rail 80 may have a larger radius (e.g. the radius of the circular arc which best approximates the curve of the guide rail at that point) than the second rear guide rail 81.

In further embodiments, the guide rails may include a linear portion and a curved portion allowing the implant 10 to expand linearly and angularly while expanding from the collapsed position to the expanded position. The expansion profile of the implant 10 may be further customized by altering the shape of the guide rails and guide grooves as needed.

In further embodiments, the guide rails may be linear. In this embodiment, an angular expansion profile may be accomplished by altering the lengths of the pin slots 82, 83, 84. As discussed in further detail below, when a pin 19 is inserted into a pin aperture 42, 43, 45 and into a pin slot 82, 83, 84, the pin 19 may bottom out against the bottom of the pin slot 82, 83, 84, thereby preventing the implant 10 from over expanding. As shown in FIG. 10, in some embodiments, the first pin slot 82 is longer than the second pin slot 83. Therefore, when the pins 19 are bottomed out against the bottom of the pin slots 82, 83, such as the embodiment shown in FIG. 5, the first lateral side portion 86 will be expanded a greater distance than the second lateral side portion 88. Therefore, an implant may include a linear guide rail on a first lateral side having a pin slot and a linear guide rail on a second lateral side. In this embodiment, if the pin slot on the first lateral side is longer than the pin slot on the second lateral side, the first lateral side will be able to expand a further distance than the second lateral side before the pins bottom out in the pin slots, thereby creating an angular expansion profile.

In this example embodiment, the angular expansion profile may be customized based on how many radians the control shaft 16 is turned in the second direction (e.g. counter-clockwise). As the control shaft 16 is turned in the second direction (e.g. counter-clockwise), the magnitude of the angle that the top surface 66 of the adjustable member 14 and the bottom surface 28 of the base member 12 will increase. Therefore, according to this example embodiment, there is a direct relationship between the number of radians the control shaft 16 is turned in the second direction (e.g. counter-clockwise) and the magnitude of the angle between the top surface 66 of the adjustable member 14 and the bottom surface 28 of the base member 12 of the implant 10 when the implant 10 is in a second, expanded position.

The magnitude of the angle that the top surface 66 of the adjustable member 14 and the bottom surface 28 of the base member 12 may also be customized by changing the curvature of the guide rails 78, 79, 80, 81 and the curvature of the guide grooves 52, 53, 54, 55. For example, adjusting the radii (e.g. the radius of the circular arc which best approximates the curve of the guide groove at that point) of the guide grooves 52, 53, 54, 55 and the radii (e.g. the radius of the circular arc which best approximates the curve of the guide rail at that point) of the guide rails 78, 79, 80, 81 will cause a change in the angular expansion profile of the implant 10. For example, decreasing the radii (e.g. the radius of the circular arc which best approximates the curve of the guide groove at that point) of the guide grooves 52, 53, 54, 55 and the radii (e.g. the radius of the circular arc which best approximates the curve of the guide rail at that point) of the guide rails 78, 79, 80, 81 will result in a larger angle between the top surface 66 of the adjustable member 14 and the bottom surface 28 of the base member 12. Therefore, according to this example embodiment, there is an inverse relationship between magnitude of the radii of the guide rails 78, 79, 80, 81 and the magnitude of the angle between the top surface 66 of the adjustable member 14 and the bottom surface 28 of the base member 12 of the implant 10 when the implant 10 is in a second, expanded position.

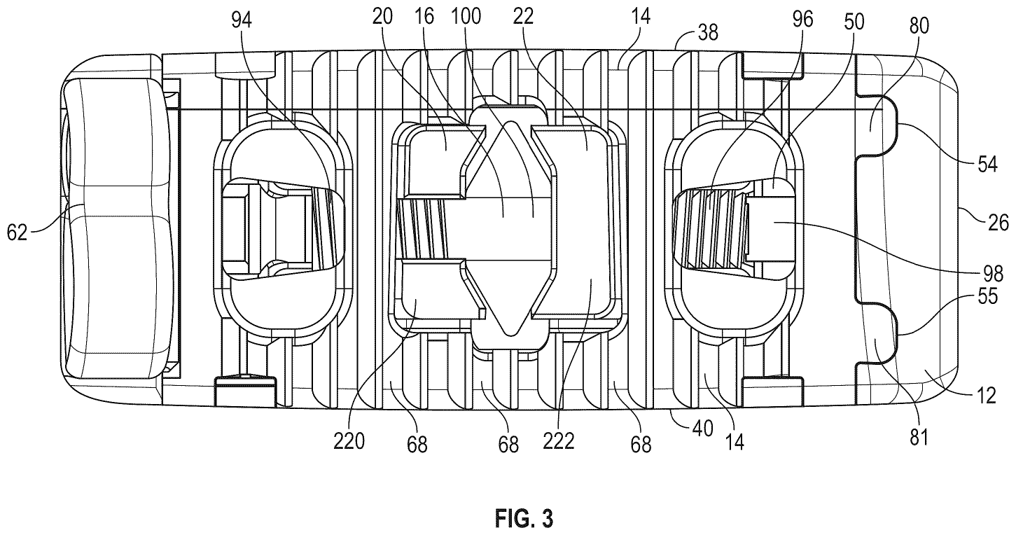

In an example embodiment, as the implant 10 expands in an angular fashion, as shown in FIGS. 5-8, the control shaft 16 will not necessarily be centered in the implant 10. For example, as shown in FIG. 3, the tip 98 is centered in the tip bore 50 when the implant 10 is in the collapsed position. However, as shown in FIG. 7, when the implant 10 is in the expanded position, the tip 98 of the control shaft 16 is no longer centered in the tip bore 50, but is instead offset.

In use, the implant 10 is positioned within a desired space (e.g., between adjacent portions of bone) while in the first, collapsed position, as shown in FIG. 1. To position the implant 10, an appropriate tool may be used to engage tool recesses 56 and to manipulate the implant 10 into a desired position. Once in a desired position, a subsequent tool may be utilized to engage the tool port 92 and to rotate the control shaft 16 to move the adjustable member 14 to a desired degree of expansion. It should be noted that based on a particular application, the adjustable member 14 may be utilized in a fully collapsed position, a fully expanded position, or any intermediate position therebetween. Once the implant 10 is properly positioned and expanded to a desired height, bone graft material may be inserted into the central cavity 36. The various apertures in and through the base member 12 and the adjustable member 14 may in some embodiments facilitate the growth of bone material in and around the implant 10 to further stabilize the device.

Once the implant 10 is positioned within a desired space and expanded to a desired degree of expansion, the control shaft 16 can be secured using a cam screw 18. The cam screw 18 includes a threaded shaft 181 and a head 182, as shown in FIG. 9. The threaded shaft 181 allows the cam screw 18 to be screwed into the base member 12. The head 182 includes a tool port 184. The tool port 184 allows the cam screw to be tightened or loosened using a tool, such as a hex head driver. While this example embodiment shows the hex head tool port 184, it should be appreciated that the tool port 184 can be designed to receive several different types of hand tools, including a slotted screw driver, a Phillips-head screwdriver, an Allen wrench screwdriver, a hexagonal drive, a torx drive, a Robertson drive, a tri-wing screwdriver, an Allen security driver, a torx security driver, a Pozidriv, a clutch drive, a spanner, a Schrader drive, a nut driver, a hex wrench, a node security driver, any combination of the listed driver interfaces, and any other type of driver interface.

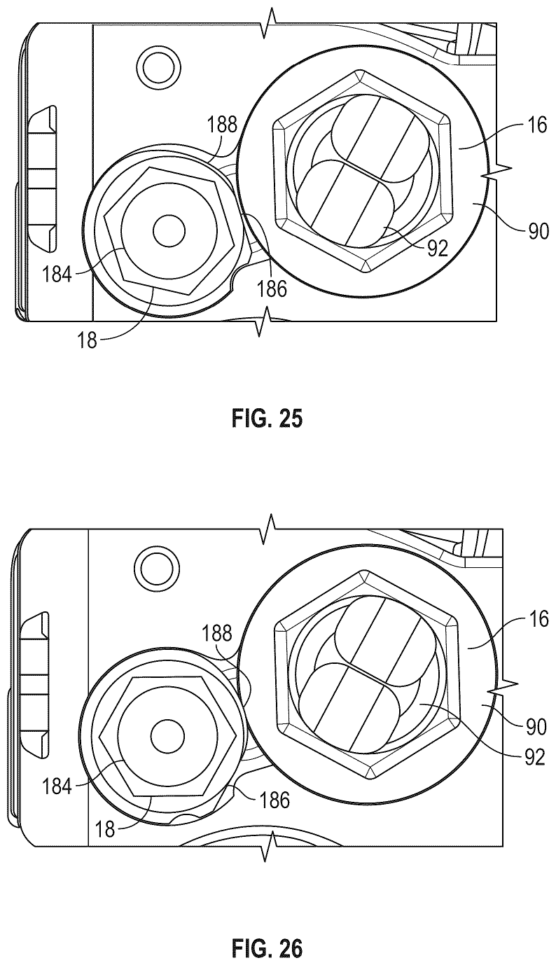

The head 182 further includes a flat portion 186 and a cam portion 188, as shown in FIGS. 25 and 26. The flat portion 186 includes a flat edge at the edge of the head 182, and the cam portion 188 includes a rounded edge with an increasing radius (e.g. the radius of the circular arc which best approximates the curve of the guide groove at that point) in the counter-clockwise direction.

In an example embodiment, when the implant 10 is in a collapsed position, the flat portion 186 of the cam screw 18 may be proximate to the control shaft 16, as shown in FIG. 25. Once the implant 10 is positioned within a desired space and expanded to a desired degree of expansion, the control shaft 16 may be locked into position by turning the cam screw 18. For example, the cam screw 18 may be tightened such that the cam portion 188 engages, and is in contact with, the head 90 of the control shaft, as shown in FIG. 26. In doing so, the force applied by the cam portion 188 of the cam screw 18 to the head 90 of the control shaft 16 may assist in preventing the control shaft 16 from rotating, thereby locking the control shaft 16 into position.

In some example embodiments, the cam screw 18 may further be configured to lock the control shaft 16 into place. In one example, after the implant 10 is installed, the implant 10 may be prone to over-expanding. In this example, as the implant 10 expands, the control shaft 16, as shown in FIG. 26, will turn in a counter-clockwise direction. In this example, the cam screw 18 may be threaded using a right-hand configuration, wherein turning the cam screw 18 in a clockwise direction will tighten the cam screw 18 into the base member 12 of the implant 10. In this example, the cam portion 188 is engaged with the head 90 of the control shaft 16. If the control shaft 16 begins to rotate in a counter-clockwise direction, the friction between the cam portion 188 and the head 90 will cause the cam screw 18, as shown in FIG. 25, to turn in a clockwise direction, further tightening the cam screw 18, which will further lock the control shaft 16 into position.

Additionally, in the example that the implant 10 is prone to over-expanding, a plurality of retention pins 19 may be utilized to prevent over-expansion. For example, as shown in FIG. 1, a retention pin 19 may be press fit into the first pin aperture 42, extending into the first pin slot 82 (see FIG. 10). In this example, when the implant 10 is in the fully expanded position, the retention pin 19 may bottom out against the bottom of the first pin slot 82, preventing the implant 10 from further expanding. Similarly, a retention pin 19 may be press fit into the second pin aperture 43, extending into the second pin slot 83. In this example, when the implant 10 is in the fully expanded position, the retention pin 19 may bottom out against the bottom of the second pin slot 83, preventing the implant 10 from further expanding. Further, a retention pin 19 may be press fit into the third pin aperture, extending into the third pin slot. In this example, when the implant 10 is in the fully expanded position, the retention pin 19 may bottom out against the bottom of the third pin slot, preventing the implant 10 from further expanding. Additionally, a retention pin 19 may be press fit into the fourth pin aperture 45, extending into the fourth pin slot 84. In this example, when the implant 10 is in the fully expanded position, the retention pin 19 may bottom out against the bottom of the third pin slot, preventing the implant 10 from further expanding.

Additionally, the expansion profile of the implant 10 may be further customized by varying the length of the pin slots 82, 83, 84. As discussed above, when a pin 19 is inserted into a pin aperture 42, 43, 45 and into a pin slot 82, 83, 84, the pin 19 may bottom out against the bottom of the pin slot 82, 83, 84, thereby preventing the implant 10 from over expanding. As shown in FIG. 10, the first pin slot 82 is longer than the second pin slot 83. Therefore, when the pins 19 are bottomed out against the bottom of the pin slots 82, 83, such as the embodiment shown in FIG. 5, the first lateral side portion 86 will be able to expanded a greater distance than the second lateral side portion 88.

In another example embodiment, after the implant 10 is installed, the implant 10 may be prone to collapsing. In this example, as the implant 10 collapses, the control shaft 16, as shown in FIG. 25, will turn in a clockwise direction. In this example, a cam screw 18 may be threaded using a left-hand configuration, wherein turning the cam screw 18 in a counter-clockwise direction will tighten the cam screw 18 into the base member 12 of the implant 10. Further, the cam screw 18 will include a cam portion 188 that will increase in radius (e.g. the radius of the circular arc which best approximates the curve of the guide groove at that point) in the clockwise direction. In this example, the cam portion 188 is engaged with the head 90 of the control shaft 16. If the control shaft 16 begins to rotate in a clockwise direction, the friction between the cam portion 188 and the head 90 will cause the cam screw 18 to turn in a counter-clockwise direction, further tightening the cam screw 18, which will further lock the control shaft 16 into position.

It should be noted that the implant 10 may share various features with the other implants described herein, and be made of the same, similar, or different materials. For example, various components of the implant 10 may be made of metal, plastic, composites, or other suitable bio-compatible materials. Further, the implant 10 may be usable in connection with the spine or other parts of the body.

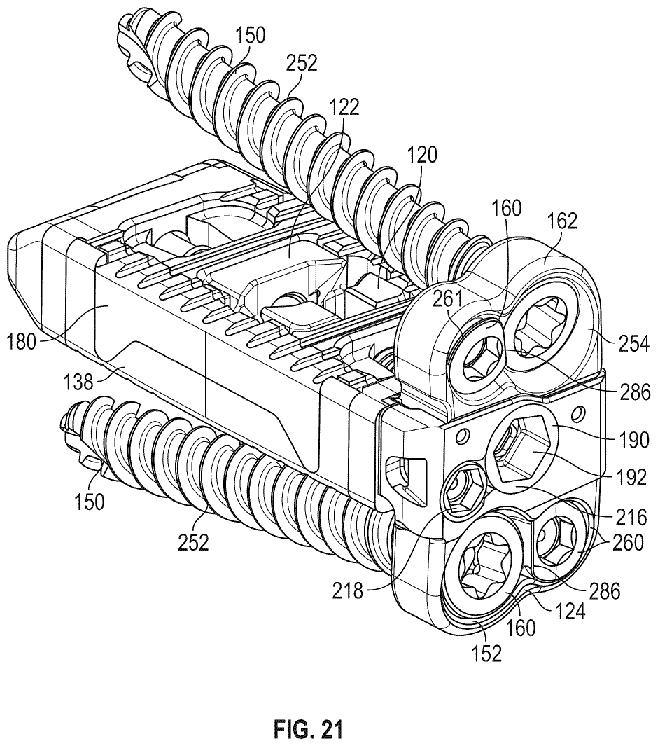

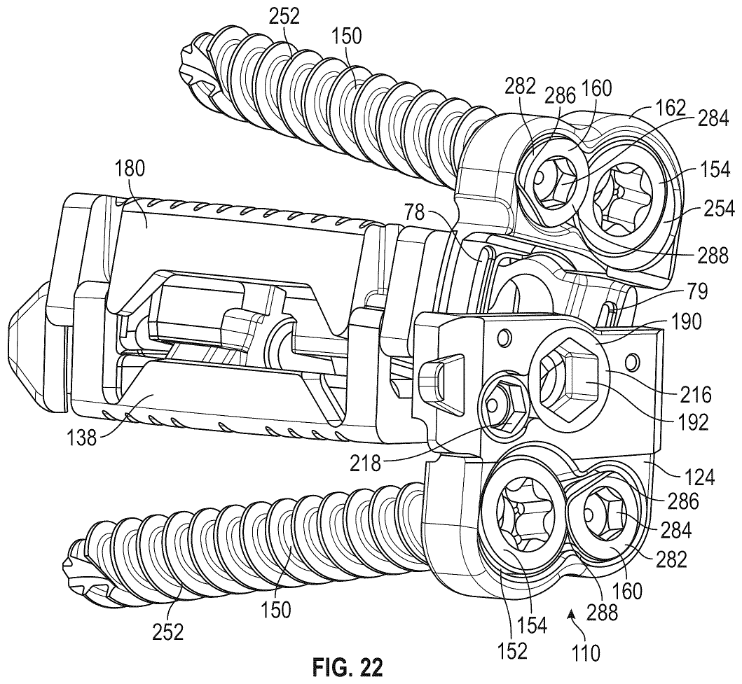

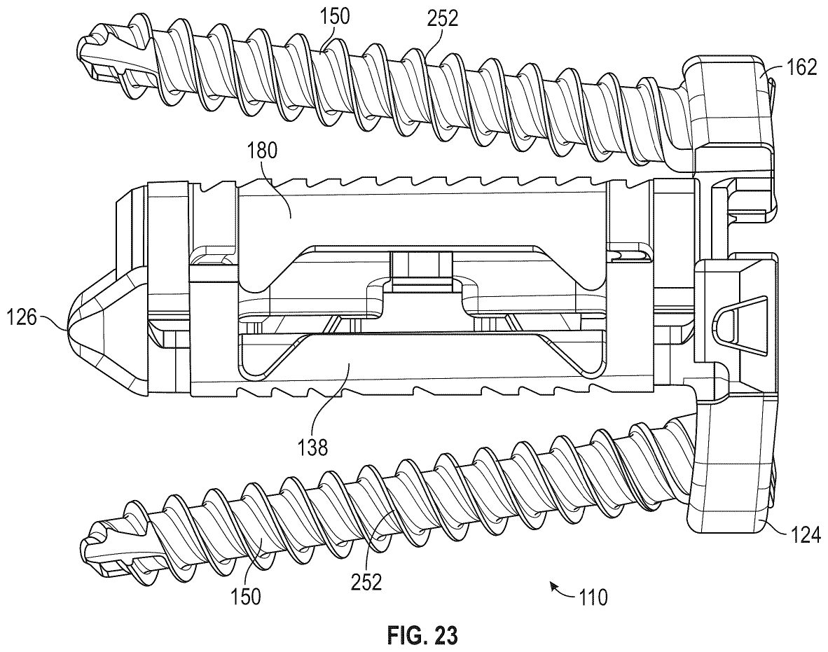

Referring now to FIGS. 21-23, in some embodiments, one or both of a base member 112 and an adjustable member 114 of an implant 110 may be configured to receive an anchoring member to further secure the implant 110 to adjacent portions of bone. In an example embodiment, the anchoring member may be a bone screw 150. For example, as shown in FIGS. 21 and 22, an implant 110 includes a base member 112 and an adjustable member 114 adjustably coupled to the base member 112. A control shaft 216 is received by the base member 112 and is retained by a cam screw 218 passing through a portion of the base member 112. A first control member 120 and a second control member 122 are received on the control shaft 216 and are movable along the control shaft 216 to adjust a position of the adjustable member 114 between a collapsed position, as shown in FIG. 21 and an expanded position, as shown in FIGS. 22 and 23. Bone screws 150 extend through base member 112 and adjustable member 114.

In some embodiments, implant 110 may share any combination of the features disclosed herein with respect to the other implants, and all such combinations of features are to be understood to be within the scope of the present disclosure. In some embodiments, the implant 110 is substantially similar to implant 10, except as described herein. In an example embodiment, the base member 112 includes a first bone screw bore 152 configured to receive bone screw 150. Similarly, the adjustable member 114 includes a second bone screw bore 254 configured to receive bone screw 150. In some embodiments, the first bone screw bore 152 is integrated into the base member 112. In some embodiments, the second bone screw bore 254 is integrated into the adjustable member 114.

According to the example embodiment shown in FIGS. 21-23, the bone screw 150 includes a linear, externally threaded shaft 252, a head 154 at a first end, and a tip 156 at a second end opposite the first end. In some embodiments, the tip 156 is pointed. In some embodiments, the diameter of the bone screw 416 remains constant from the head 154 to the tip 156. The head 154 further includes a socket 158 that is configured to receive an installation tool. While this example embodiment has a torx drive socket 158, it should be appreciated that the socket 158 can be designed to receive several different types of hand tools, including a slotted screw driver, a Phillips-head screwdriver, an Allen wrench screwdriver, a hexagonal drive, a torx drive, a Robertson drive, a tri-wing screwdriver, an Allen security driver, a torx security driver, a Pozidriv, a clutch drive, a spanner, a Schrader drive, a nut driver, a hex wrench, a node security driver, any combination of the listed driver interfaces, and any other type of driver interface.

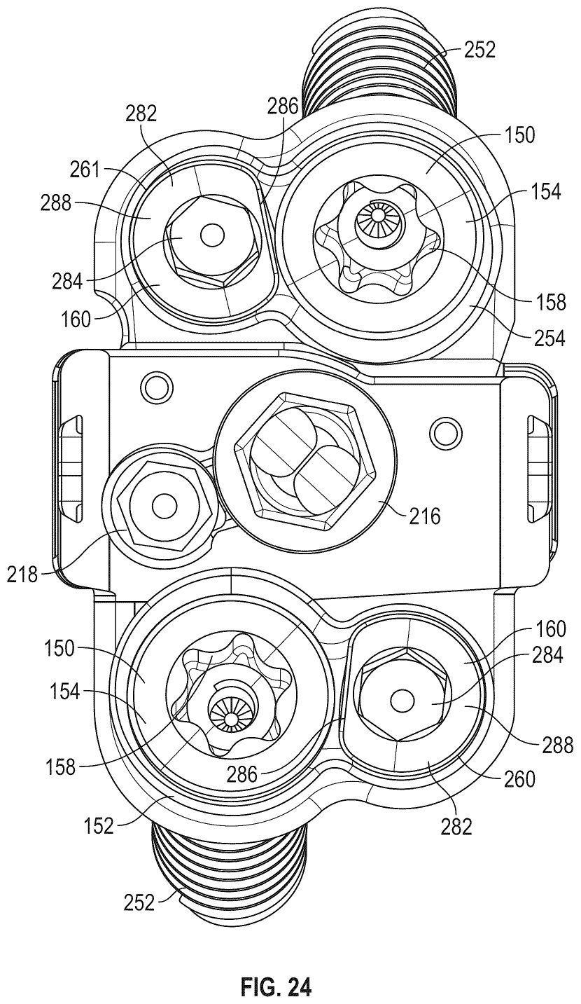

Once the bone screw 150 is inserted into a bone, a retention screw 160 may be used to prevent a back-out of the bone screw 150. In an example embodiment, such as the embodiment shown in FIG. 24, the retention screw 160 may include a head 282, a tool port 284, and a threaded shaft. The threaded shaft may be screwed into a first threaded bore 260 in the base member 112 or into a second threaded bore 261 in the adjustable member 114, as shown in FIGS. 21-24. In some embodiments, the first threaded bore 260 is integrated into the base member 112. In some embodiments, the second threaded bore 261 is integrated into the adjustable member 114.

The head 282 further includes a flat portion 286 and a rounded shoulder portion 288. In some embodiments, when the flat portion 286 is proximate the head 154 of the bone screw, the retention screw 160 is not in contact with the bone screw 150, as shown in FIG. 24. However, the retention screw 160 may be tightened into the first threaded bore 260 or the second threaded bore 261, such that the rounded shoulder portion 228 is proximate to the bone screw 150. In some embodiments, when the retention screw 160 is tightened into the first threaded bore 260 or second threaded bore 261, the underside of the rounded shoulder portion 228 is in contact with the head 154 of the bone screw 150. In doing so, the retention screw 160 may be used to prevent back out of the bone screw 150.

In the example embodiment shown in FIGS. 21-24, the retention screw 160 includes a tool port 284 configured to receive a hex head driver. It should be appreciated that the tool port 284 can be designed to receive several different types of hand tools, including a slotted screw driver, a Phillips-head screwdriver, an Allen wrench screwdriver, a hexagonal drive, a torx drive, a Robertson drive, a tri-wing screwdriver, an Allen security driver, a torx security driver, a Pozidriv, a clutch drive, a spanner, a Schrader drive, a nut driver, a hex wrench, a node security driver, any combination of the listed driver interfaces, and any other type of driver interface.

Referring now to the Figures generally, the various embodiments disclosed herein provide expandable implants including a base member, an adjustable member adjustably coupled to the base member and movable between a first, collapsed position, and a second, expanded position, and a control shaft rotatably received by the base member, where rotation of the control shaft causes relative movement of the adjustable member relative to the base member. At least one control member is received on the control shaft and by the control channel, and rotation of the control shaft causes the control member to translate along the control shaft and along the control channel.

In some embodiments, the top surface of the upper support and the bottom surface of the bottom support are parallel when the implant is in the first, collapsed position (see e.g., FIG. 1). However, in other embodiments, the top surface of the upper support and the bottom surface of the lower support may form an angle when the implant is in the first, collapsed position. In this example embodiment, the implant will have a larger height (i.e., the distance between the top surface of the upper support and the bottom surface of the lower support) on one lateral side of the implant than the other lateral side of the implant. In this embodiment, the implant may expand linearly (i.e., the angle remains constant as the implant expands), or the implant may angularly expand (i.e., the angle increases as the implant expands).

In some embodiments, the adjustable member moves in a linear fashion relative to the base member. In other embodiments, the adjustable member moves in a non-linear fashion relative to the base member. In further embodiments, the adjustable member pivots about a pivot axis relative to the base member. The pivot axis may be provided by a pivot pin extending through one or both of the adjustable member and the base member.

In some embodiments, a single control member and control channel are utilized. In other embodiments, multiple (e.g., 2) control members and control channels are utilized. In some embodiments, the multiple control channels are parallel and straight. In other embodiments, the control channels are non-parallel and straight (e.g., angled toward each other). In further embodiments, the control channels are non-parallel and non-straight such that the adjustable member moves in a non-linear fashion relative to the base member.

In some embodiments, the control shaft includes a control thread corresponding to each control member. As such, while in some embodiments the control shaft includes a single control thread, in other embodiments the control shaft includes multiple (e.g., first and second) control threads. In some embodiments, the control threads are like-threaded. In other embodiments, the control threads have different threads. For example, in some embodiments, a first control thread is opposite-handed from a second control thread. In further embodiments, a first control thread has a different pitch from a second control thread. In yet further embodiments, a first control thread is different handed and has a different pitch from a second control thread.

In some embodiments, one or both of the adjustable member and the base member include projections/grooves to provide a gripping surface intended to facilitate gripping adjacent portions of bone. In further embodiments, one or both of the adjustable member and the base member include one or more apertures and/or cavities configured to promote bone growth in and around the adjustable member and the base member. In some embodiments, the apertures extend from a top, bottom, and/or side surface of the adjustment member or the base member and to a central cavity of the implant.

According to any of the embodiments disclosed herein, one or more bone screws may be included and positioned to extend through one or both of the adjustable member and the base member and into adjacent portions of bone. In some embodiments, multiple bone screws are used. A first bone screw may extend through the adjustable member and into a first portion of bone, and a second bone screw may extend through the base member and into a second portion of bone. In further embodiments, multiple bone screws are accessible and manipulatable by way of the front face of the implant defined by one or both of the adjustable member and the base member. A head and tool port of the control shaft may further be accessible by way of the front face of the implant.

In various embodiments, any suitable configuration of the control shaft/control member(s)/control channel(s) may be utilized. In some embodiments, an at least partially spherical control member threadingly engages a threaded control shaft and translates both along the control shaft and within the control channel. In other embodiments, the control member is non-spherical and is received at least partially on or in a control rail or control channel provided by the adjustable member, such that the control member translates along both the control shaft and the control channel or control rail.

It is important to note that the construction and arrangement of the elements of the various implants and implant components as shown in the exemplary embodiments are illustrative only. Although a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, materials, colors, orientations, etc.) without materially departing from the novel teachings and advantages of the subject matter recited in the various embodiments. Accordingly, all such modifications are intended to be included within the scope of the present disclosure as defined in the appended claims. The order or sequence of any process or method steps may be varied or re-sequenced according to alternative embodiments. Other substitutions, modifications, changes, and/or omissions may be made in the design, operating conditions, and arrangement of the exemplary embodiments without departing from the spirit of the present disclosure.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of some features described and claimed without restricting the scope of these features to the precise numerical ranges provided. Accordingly, these terms should be interpreted as indicating that insubstantial or inconsequential modifications or alterations of the subject matter described and claimed are considered to be within the scope of the application as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on hardware systems chosen and on designer choice. All such variations are within the scope of the disclosure.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present application.

It should be appreciated that dimensions of the components, structures, and/or features of the present implants and installation instruments may be altered as desired within the scope of the present disclosure.

What is claimed is:

1. An expandable implant comprising:
a base member having a bottom surface;
an adjustable member adjustably coupled to the base member and movable between a first, collapsed position, and a second, expanded position, wherein the adjustable member has a top surface;
a first lateral side extending along a length of the expandable implant and having a first height defined by the top surface of the adjustable member and the bottom surface of the base member proximate the first lateral side;
a second lateral side opposite the first lateral side, extending along the length of the expandable implant and having a second height defined by the top surface of the adjustable member and the bottom surface of the base member proximate the second lateral side;
wherein a change in the first height proximate the first lateral side is different than a change in the second height proximate the second lateral side when the expandable implant is moved from the first, collapsed position to the second, expanded position, and
wherein the change in the first height at the first lateral side and the change in the second height at the second lateral side are each greater than zero.

2. The expandable implant of claim 1, wherein the first height proximate the first lateral side is substantially equal to the second height proximate the second lateral side when the expandable implant is in the first, collapsed position.

3. The expandable implant of claim 1, further comprising a control assembly comprising a control shaft and a control member threadingly received on the control shaft, and wherein rotation of the control shaft causes relative movement of the adjustable member relative to the base member.

4. The expandable implant of claim 3, wherein the control member comprises a first control member received on the control shaft and configured to be received within a first control channel on the adjustable member.

5. The expandable implant of claim 4, wherein the control member comprises a second control member received on the control shaft and configured to be received within a second control channel on the adjustable member.

6. The expandable implant of claim 5, wherein the first control member moves towards the second control member along the control shaft in response to the control shaft being turned in a first direction; and
wherein the first control member moves away from the second control member along the control shaft in response to the control shaft being turned in a second direction.

7. The expandable implant of claim 3, wherein the control shaft is secured using a cam screw, and wherein the cam screw has a cam portion configured to engage the control shaft.

8. An expandable implant comprising:
a first member;
a second member adjustably coupled to the first member and movable between a first, collapsed position, and a second, expanded position;
wherein the second member moves in a non-linear manner relative to the first member from the first, collapsed position to the second, expanded position such that a change in a height proximate a first lateral side of the expandable implant is different than a change in a height proximate a second lateral side of the expandable implant opposite the first lateral side, and
wherein the change in the height at the first lateral side and the change in the height at the second lateral side are each greater than zero.

9. The expandable implant of claim 8, wherein the first member comprises a top surface and a bottom surface opposite the top surface;
wherein the second member has a top surface and a bottom surface opposite the top surface; and
wherein the bottom surface of the first member is substantially parallel to the top surface of the second member in the first, collapsed position.

10. The expandable implant of claim 9, wherein the bottom surface of the first member forms a first angle with the top surface of the second member in the second, expanded position, wherein the first angle is greater than 0 degrees.

11. The expandable implant of claim 8, further comprising a control assembly comprising a control shaft and a control member, wherein rotation of the control shaft causes relative movement of the second member relative to the first member.

12. The expandable implant of claim 11, wherein the control member comprises a first control member received on the control shaft and configured to be received within a first control channel on the second member.

13. The expandable implant of claim 12, further comprising a second control member received on the control shaft and configured to be received within a second control channel on the second member.

14. The expandable implant of claim 13, wherein the first control member moves towards the second control member along the control shaft in response to the control shaft being turned in a first direction; and
wherein the first control member moves away from the second control member along the control shaft in response to the control shaft being turned in a second direction.

15. An expandable implant comprising:
a base member comprising a first body portion and a first transverse plate portion configured to receive a first anchoring member;
an adjustable member comprising a second body portion and a second transverse plate portion configured to receive a second anchoring member, wherein the adjustable member is adjustably coupled to the base member, and wherein the adjustable member moves non-linearly with respect to the base member between a first position and a second position; and
a control assembly configured to cause movement of the adjustable member relative to the base member;
wherein a change in a height proximate a first lateral side of the expandable implant is different than a change in a height proximate a second lateral side of the expandable implant opposite the first lateral side, and
wherein the change in the height proximate the first lateral side and the change in the height proximate the second lateral side are each greater than zero.

16. The expandable implant of claim 15,
wherein the adjustable member has a top surface and the base member has a bottom surface;
wherein the top surface of the adjustable member and the bottom surface of the base member define an implant height; and
wherein the implant height proximate the first lateral side is greater than the implant height proximate the second lateral side when the expandable implant is in the second position.

17. The expandable implant of claim 16, wherein the top surface of the adjustable member and the bottom surface of the base member are substantially parallel in the first position.

18. The expandable implant of claim 15, wherein the control assembly includes a first control member received on a control shaft and configured to be received within a first control channel on the adjustable member.

19. The expandable implant of claim 15, wherein the first anchoring member is secured using a retention screw.

20. The expandable implant of claim 19, wherein the retention screw has a flat portion and a rounded shoulder portion.
